(19) United States
(12) Patent Application Publication
(10) Pub. No.: US 2014/0163067 A1
(43) Pub. Date: Jun. 12, 2014

(54) Methods of Treatment of Limited Cognitive Impairment

(71) Applicant: Gerhard Koenig, Newton, MA (US)

(72) Inventor: Gerhard Koenig, Newton, MA (US)

(73) Assignee: ENVIVO PHARMACEUTICALS, INC., Watertown, MA (US)

(21) Appl. No.: 14/130,081

(22) PCT Filed: Jun. 28, 2012
(86) PCT No.: PCT/US12/44589

§ 371 Date: Feb. 27, 2014

Related U.S. Application Data
(60) Provisional application No. 61/504,154, filed on Jul. 1, 2011.

(51) Int. Cl. C07D 453/02

(52) U.S. Cl. CPC C07D 453/02
USPC 514/305

(57) ABSTRACT

A method for improving or treating a Limited Cognitive Impairment (LCI) comprising administering to a subject a compound of the invention, or a pharmaceutically acceptable salt thereof is described together with related compositions.

### CROSS REFERENCE TO RELATED APPLICATION

This application claims the benefit of priority from U.S. Provisional Application No. 61/504,154, filed Jul. 1, 2011. The foregoing related application, in its entirety, is incorporated herein by reference.

### BACKGROUND OF THE INVENTION

Nicotinic acetylcholine receptors (nAChR) form a family of ion channels activated by acetylcholine. Functional receptors contain five subunits, and there are numerous receptor subtypes. Studies have shown that central nicotinic acetylcholine receptors are involved in learning and memory. Nicotinic acetylcholine receptors of the alpha7 subtype are prevalent in the hippocampus and cerebral cortex.

WO 03/055878, published in 2003, describes a variety of agonists of the alpha7 nAChR said to be useful for improving cognition. WO 03/055878 suggests that certain agonists of the alpha7 nAChR are useful for improving perception, concentration, learning or memory, especially cognitive impairments like those occurring for example in conditions/diseases/syndromes such as mild cognitive impairment, age-associated learning and memory impairments, age-associated memory loss, Alzheimer's disease (AD), schizophrenia and certain other cognitive disorders.

In the years following this publication there have been numerous additional publications that have continued to develop the area related to therapy for improving cognition; however, there is no standard of treatment on the market today that would satisfy the current need for effective treatment of cognitive disorders. Moreover, there are no current therapies that address cognitive issues, i.e., symptoms or conditions, which precede AD on a clinical diagnostic scale but are not quantitatively or qualitatively classified as mild cognitive impairment. As such, there is a desperate need for additional therapies useful for improving cognition and/or treating cognitive disorders.

### SUMMARY OF THE INVENTION

The present invention provides methods for improving cognition, learning or memory and/or treating disorders or impairments of cognition, learning or memory that fall within the scope, or classification of Limited Cognitive Impairment (LCI) as described herein. In particular, the compounds of the invention, e.g., a compound of formula (I), have been found to improve cognition and/or treat cognitive disorders that are classified as a Limited Cognitive Impairment (LCI). Furthermore, the present invention also provides specific compositions, e.g., pharmaceutical compositions, comprising a compound of the invention useful for improving or treating Limited Cognitive Impairment (LCI).

Accordingly, in one aspect the invention provides a method for improving and/or treating a Limited Cognitive Impairment (LCI) comprising administering to a subject a therapeutically effective amount of a compound of formula (I), or a pharmaceutically acceptable salt thereof,

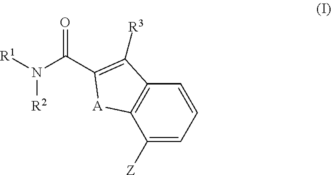

wherein

R¹ is 1-azabicyclo[2.2.2]oct-3-yl,

R² is hydrogen or (C₁-C₆)alkyl,

R³ is hydrogen, halogen or (C₁-C₆)alkyl,

A is oxygen or sulfur, and

Z is halogen, formyl, carbamoyl, cyano, trifluoromethyl, trifluoromethoxy, nitro, amino, formamido, acetamido, (C₁-C₆)alkyl, (C₁-C₆)alkoxy, (C₁-C₆)alkylthio, (C₁-C₆)alkylamino, heteroaryl-carbonylamino, arylcarbonylamino, (C₁-C₄)alkylsulfonylamino, di(arylsulfonyl)amino, (C₃-C₆)cycloalkylcarbonylmethyl or amino(hydroxyimino)methyl.

In another aspect, the invention provides a pharmaceutical composition comprising a therapeutically effective amount of (R)-7-chloro-N-(quinuclidin-3-yl)benzo[b]thiophene-2-carboxamide, or a pharmaceutically acceptable salt thereof, useful for improving or treating LCI in a subject.

In yet another aspect, the invention provides a packaged pharmaceutical comprising a package containing a unit dosage pharmaceutical composition comprising (R)-7-chloro-N-(quinuclidin-3-yl)benzo[b]thiophene-2-carboxamide or a pharmaceutically acceptable salt thereof, and instructions for use in improving or treating LCI.

### DETAILED DESCRIPTION OF THE INVENTION

The present invention provides methods for improving cognition, learning or memory and/or treating disorders or impairments of cognition, learning or memory that fall within the scope, or classification of Limited Cognitive Impairment (LCI) as described herein. The present invention, including methods of treatment, methods of preparation, and pharmaceutical compositions will be described with reference to the following definitions that, for convenience, are set forth below. Unless otherwise specified, the below terms used herein are defined as follows:

### I. DEFINITIONS

For purposes of the present disclosure, the following definitions will be used (unless expressly stated otherwise).

As used herein, “cognition” or “cognitive function” refers to the process of thought. The expressions “cognitive disorder” or a “cognitive impairment” refer to a chronic or temporary deficiency in cognition: for example, a subnormal functioning in one or more cognitive aspects such as memory, intellect, or learning and logic ability, in a particular individual relative to other individuals within the same general age population or to a non-impaired individual. This also includes any reduction in any particular individual's functioning in one or more cognitive aspects.

The language “Limited Cognitive Impairment,” “limited cognitive impairment,” and “LCI” are used interchangeably herein, and describe cognitive impairment (i.e., symptoms or conditions), which precede AD on a clinical diagnostic scale but are not of such an increased degree to be quantitatively or qualitatively classified as mild cognitive impairment; and includes any chronic or temporary impairment in cognition, learning or memory that prevents or reduces the ability of a particular individual from achieving their individual potential capacity in these areas. For example, LCIs may include minor impairments to memory associated with focus and concentration (e.g., accuracy and speed of learning and recalling information), working memory (e.g., used in decision making and problem solving), cognition, focus, mental quickness, and mental clarity. In certain embodiments, the dose of a compound of the invention would be less than the dose to treat disorders/conditions that fall within the scope of mild cognitive impairment, AD, or any condition in between the two on the clinical diagnostic scale.

In particular, limited cognitive impairment, as described herein, does not include dementia, for example Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; Alzheimer's related dementia; delirium; amnestic disorder; post-traumatic stress disorder; mental retardation; a learning disorder, for example reading disorder, mathematics disorder, post operative cognitive decline, or a disorder of written expression; attention-deficit/hyperactivity disorder; age-related cognitive decline; and any cognitive impairments resulting from chemical dependency.

An individual with “mild cognitive impairment” is an individual who meets the following clinical criteria of amnestic MCI (Petersen et al. Arch Neurol 56:303-308 (1999): 1) memory complaints corroborated by an informant, 2) objective memory impairment for age and education, 3) normal general cognitive function, 4) intact activities of daily living, and 5) the subject does not meet criteria for dementia. Mild cognitive impairment, or pre-senile dementia, is characterized by memory impairment rather than attention deficit problems, and with otherwise unimpaired cognitive functioning. Mild cognitive impairment may also be distinguished from senile dementia in that mild cognitive impairment involves a more persistent and troublesome problem of memory loss for the age of the subject. On the clinical diagnostic scale, mild cognitive impairment is followed in increased severity by early AD, and then late stage AD.

An “individual with moderate (early) AD (EAD)” is an individual who demonstrate the following criteria: 1) a decline in cognitive function for a previous higher level, 2) declines in one or more areas of cognition in addition to memory, 3) a clinical dementia rating scale score of 0.5 to 1, and 4) a clinical examination that excluded other causes of dementia.

An “individual with severe (late stage) AD (LAD)” is an individual who meets the standard clinical diagnostic criteria for probable AD (McKhann et al. Neurology 34:939-48 (1984).

The terms “treating”, “treatment”, or the like, as used herein covers the treatment of a disease-state (i.e., disorder, symptom, or condition) in an animal that is related to LCI, and includes at least one of: (i) preventing the disease-state from occurring, in particular, when such animal is predisposed to the disease-state but has not yet developed symptoms of having it; (ii) inhibiting the disease-state, i.e., partially or completely arresting its development; (iii) relieving the disease-state, i.e., causing regression of symptoms of the disease-state, or ameliorating a symptom of the disease-state; and (iv) reversal or regression of the disease-state, preferably eliminating or curing of the disease-state. In a preferred embodiment the terms “treating”, “treatment”, or the like, covers the treatment of a disease-state in an animal and includes at least one of (ii), (iii) and (iv) above. In a preferred embodiment of the present disclosure the animal is a mammal, preferably a primate, more preferably a human. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

The language “improving Limited Cognitive Impairment,” as in a method of improving Limited Cognitive Impairment” describes the improvement of the symptoms manifested by the underlying LCI.

The expression “therapeutically effective,” as used herein, describes amounts or doses of a compound useful for improving or treating of a disorder or symptom falling within the scope of Limited Cognitive Impairments (LCI), alone or in combination with other compounds/compositions, that are therapeutically effective for the purpose for which they were intended and achieve a therapeutic effect, e.g., improving cognition. In certain embodiments, to achieve a therapeutic effect, doses or amounts are provided by a well-regulated or well-designed regimen of administration. As such, therapeutically effective amounts or doses include amounts or doses that would not otherwise be therapeutically effective alone (i.e., in the absence of the combinations of the present invention), or what might otherwise be referred to as a subclinical dose. The amount needed to elicit the therapeutic response can be determined based on the age, health, size and sex of the patient. Optimal amounts can also be determined based on monitoring of the patient's response to treatment. Administration may be by any route, including, without limitation, parenteral, oral, sublingual, transdermal, topical, intranasal, intratracheal, or intrarectal. In certain preferred embodiments, administration of the compounds may preferably be by the oral route.

As used herein, the terms “subject”, and “patient” are used interchangeably. The terms “subject” and “patient” refer to an animal (e.g., a bird such as a chicken, quail or turkey) or a mammal including non-primates (e.g., a cow, pig, horse, sheep, rabbit, guinea pig, rat, cat, dog, and mouse) and primates (e.g., a monkey, chimpanzee and a human). In a particular embodiment, the subject is a human.

As used herein, “alkyl” refers to a linear or branched saturated or unsaturated aliphatic C₁-C₆ hydrocarbon, unless some other number of carbon atoms is specified. Unsaturation in the form of a double or triple carbon-carbon bond may be internal or terminally located and, in the case of a double bond, both cis and trans isomers are included. An optionally substituted alkyl can be independently substituted with one or more substituents selected from halogen, e.g., F, oxo, OH, (C₁-C₄)alkoxy, (C₃-C₆)cyclo alkyloxy, (C₁-C₄)alkylthio, (C₃-C₆)cycloalkylthio-, —C(O)NH₂, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl-, —S(O)₂NH₂, —S(O)₂NH(C₁-C₄)alkyl, —S(O)₂N[(C₁-C₄)alkyl(C₁-C₄)alkyl]. Examples of alkyl groups include, but are not limited to, methyl, ethyl, n-propyl, isopropyl, t-butyl, n-pentyl, n-hexyl, isobutyl, neopentyl, cis- and trans-2-butenyl, isobutenyl and propargyl. C₁-C₄ alkyl is the subset of alkyl limited to a total of up to 4 carbon atoms.

In each case in which a size range for the number of atoms in a ring or chain is disclosed, all subsets are disclosed. Thus, C_{X}-C_{y} includes all subsets, e.g., C₁-C₄ includes C₁-C₂, C₂-C₄, and C₁-C₃ as well as C₁, C₂, C₃ and C₄.

As used herein “alkoxy” refers to an alkyl-O— group wherein alkyl is as defined above. C₁-C₆ alkoxy is the subset of alkyl-O— where the subset of alkyl is limited to a total of up to 6 carbon atoms. Examples of alkoxy groups include methoxy, trifluoromethoxy, ethoxy, trifluoroethoxy, and propoxy.

As used herein, “alkylthio” refers to an alkyl-S— group wherein alkyl is as defined above. C₁-C₆ alkylthio is the subset of alkyl-S— where the subset of alkyl is limited to a total of up to 6 carbon atoms.

As used herein, “alkylamino” refers to alkyl-NH— wherein alkyl is as defined above.

As used herein, the term “aryl” means a mono- or polycyclic hydrocarbon, containing from 6 to 15 carbon atoms, in which at least one ring is aromatic. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Aryl groups included in compounds of this invention may be optionally substituted with one or more substituents. In one embodiment, the aryl group is a monocyclic ring, wherein the ring comprises 6 carbon atoms. An optionally substituted aryl can be independently substituted with one or more substituents selected from halogen, CF₃, CN, OH, (C₁-C₄)alkoxy, (C₁-C₄)alkylthio, (C₃-C₇)cycloalkylthio, (C₃-C₇)cycloalkyloxy, aryloxy, (C₁-C₄)alkoxy(C₁-C₄)alkyloxy, hetero(C₃-C₇)cycloalkyloxy, heteroaryloxy, —OC(O)Rₐ, —OC(O)NHRₐ, —OC(O)N(Rₐ)(R_{b}), —S(O)Rₐ, —NHRₐ, —N(Rₐ)(R_{b}), —NHC(O)Rₐ, —N(Rₐ)C(O)R_{b}, —NHC(O)ORₐ, —N(Rₐ)C(O)OR_{b}, —N(Rₐ)—C(O)—NH(R_{b}), —N(Rₐ)—C(O)—N(R_{b})₂, —C(O)NH₂, —C(O)NHRₐ, —C(O)N(Rₐ)(R_{b}), —CO₂H, —CO₂Rₐ, —CORₐ and R_{c} wherein Rₐ, R_{b} and R_{c} are independently chosen from (C₁-C₆)alkyl, (C₁-C₄)alkoxy(C₁-C₄)alkyl, —CH₂CH₂OH, —CH₂CH₂OMe, (C₃-C₇)cycloalkyl, (C₃-C₇)cycloalkyl(C₁-C₄)alkyl, aryl, arylalkyl, heteroaryl, heteroarylalkyl, hetero(C₃-C₇)cycloalkyl, and hetero(C₃-C₇)cycloalkyl(C₁-C₄)alkyl, each of which is optionally and independently substituted with up to three groups selected from halogen, (C₁-C₆)alkyl, (C₃-C₇)cycloalkyl, (C₃-C₇)cycloalkyloxy, (C₃-C₇)cycloalkylalkoxy, CN, CHF₂, CF₃, CH₂CF₃, NHMe, NMe₂, piperidinyl, morpholinyl, N-Me-piperazinyl, piperazinyl, OCF₃, OCHF₂, OCH₂CF₃, SMe, each of which are attached via carbon-carbon or carbon-nitrogen or carbon-oxygen single bonds, and none of which are substituted; or Rₐ and R_{b} taken together with the atom(s) to which they are attached form a 5-6 membered ring. In other cases, an optionally substituted aryl can be independently substituted with one or more substituents selected from halogen CF₃, CN, (C₁-C₆)alkyl, (C₃-C₇)cycloalkyl, (C₁-C₄)alkoxy, (C₃-C₇)cycloalkylthio, (C₃-C₇)cycloalkyloxy, hetero(C₃-C₇)cycloalkyl, C(O)NH₂, —C(O)NHRₐ, —C(O)N(Rₐ)(R_{b}) wherein Rₐ and R_{b} are defined as above. In further cases, an optionally substituted aryl can be independently substituted with one or more substituents selected from halogen CF₃, CN, (C₁-C₆)alkyl, (C₃-C₇)cycloalkyl, (C₃-C₇)cycloalkyloxy, and hetero(C₃-C₇)cycloalkyl.

As used herein “cycloalkyl” is a C₃-C₈ cyclic non-aromatic hydrocarbon which may contain a single double bond. An optionally substituted cycloalkyl can be independently substituted with one or more substituents selected from F, oxo, OH, (C₁-C₆)alkyl, (C₁-C₄)alkoxy, —(C₃-C₇)cycloalkyl, (C₃-C₇)cycloalkylalkyl, (C₃-C₇)cycloalkyloxy, (C₁-C₄)alkylthio, (C₃-C₆)cycloalkylthio-, —C(O)NH₂, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl-, —S(O)₂NH₂, —S(O)₂NH(C₁-C₄)alkyl, —S(O)₂N[(C₁-C₄)alkyl(C₁-C₄)alkyl]. In other cases, an optionally substituted cycloalkyl can be independently substituted with one or more substituents selected from F, oxo, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl-, —S(O)₂NH(C₁-C₄)alkyl, —S(O)₂N[(C₁-C₄)alkyl(C₁-C₄)alkyl]. In further cases, an optionally substituted cycloalkyl can be independently substituted with one substituent selected from oxo, OH, (C₁-C₆)alkyl, (C₁-C₄)alkoxy, (C₃-C₇)cycloalkylalkyl, (C₃-C₇)cycloalkyloxy, (C₁-C₄)alkylthio, (C₃-C₆)cycloalkylthio-, —C(O)NH₂, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl-, —S(O)₂NH₂, —S(O)₂NH(C₁-C₄)alkyl, —S(O)₂N[(C₁-C₄)alkyl(C₁-C₄)alkyl]. Examples of cycloalkyl groups include cyclopropyl, cyclobutyl, cyclopentyl, cyclohexyl, cyclopentenyl and cyclohexanonyl.

As used herein, “halogen” refers to F, Cl, Br or I. In particular embodiments, halogens are F, Cl and Br.

As used herein, the term “halo-substituted alkyl” or “haloalkyl” refers to an alkyl as defined herein which is substituted by one or more halogen atoms, or halo groups as defined herein. The haloalkyl can be monohaloalkyl, dihaloalkyl, polyhaloalkyl, or perhaloalkyl. For example, a monohaloalkyl contains one iodo, bromo, chloro or fluoro within the alkyl group. For multiple substituted haloalkyls, the halo atoms may be the same or a combination of different halo groups within the alkyl. Typically the polyhaloalkyl contains up to 6, or 4, or 3, or 2 halo groups. In a preferred embodiment, halo-substituted alkyl groups have about 1 to 6 carbons, or 1-3 carbons. Non-limiting examples of haloalkyl include fluoromethyl, difluoromethyl, trifluoromethyl, chloromethyl, dichloromethyl, trichloromethyl, pentafluoroethyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms.

As used herein, the term “heteroaryl” refers to a 5-14 membered monocyclic- or bicyclic- or tricyclic-aromatic ring system, having 1 to 8 heteroatoms selected from N, O or S. Typically, the heteroaryl is a 5-10 membered ring system (e.g., 5-7 membered monocycle or an 8-10 membered bicycle). A 5-7 membered monocyclic ring system preferably contains 1 to 3 heteroatoms each independently selected from O, N, or S. Exemplary heteroaryl groups include, but are not limited to 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, and 2-, 4-, or 5-pyrimidinyl.

As used herein the term “dose” is the amount of active pharmaceutical ingredient (API) administered to a subject. For example 1 mg means 1 mg of API was orally administered to each subject each day.

In certain embodiments, where the “API” or “Active Pharmaceutical Ingredient” is defined as a compound of formula (I), e.g., (R)-7-chloro-N-(quinuclidin-3-yl)benzo[b]thiophene-2-carboxamide, such language is intended to include pharmaceutically acceptable salts, solvates, or solvates of pharmaceutically acceptable salts. For example, the recited compound (R)-7-chloro-N-(quinuclidin-3-yl)benzo[b]thiophene-2-carboxamide includes either (R)-7-chloro-N-(quinuclidin-3-yl)benzo[b]thiophene-2-carboxamide hydrochloride, (R)-7-chloro-N-(quinuclidin-3-yl)benzo[b]thiophene-2-carboxamide, (R)-7-chloro-N-(quinuclidin-3-yl)benzo[b]thiophene-2-carboxamide hydrochloride monohydrate or (R)-7-chloro-N-(quinuclidin-3-yl)benzo[b]thiophene-2-carboxamide hydrochloride solvate. Where solvate represents a stoichiometric ratio of 0.1 to 10 molecules of solvent compared to (R)-7-chloro-N-(quinuclidin-3-yl)benzo[b]thiophene-2-carboxamide hydrochloride or (R)-7-chloro-N-(quinuclidin-3-yl)benzo[b]thiophene-2-carboxamide. Solvent molecules include but are not limited to water, methanol, 1,4 dioxane, ethanol, iso-propanol or acetone. In some cases water is the preferred solvate.

### II. COMPOUNDS OF THE INVENTION

The compounds of the invention useful in the methods described herein may be selected from a compound of formula (I), or a pharmaceutically acceptable salt thereof,

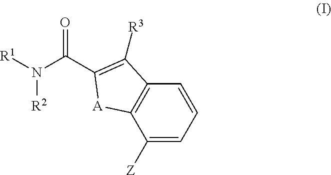

wherein

R¹ is 1-azabicyclo[2.2.2]oct-3-yl,

R² is hydrogen or (C₁-C₆)alkyl,

R³ is hydrogen, halogen or (C₁-C₆)alkyl,

A is oxygen or sulfur, and

Z is halogen, formyl, carbamoyl, cyano, trifluoromethyl, trifluoromethoxy, nitro, amino, formamido, acetamido, (C₁-C₆)alkyl, (C₁-C₆)alkoxy, (C₁-C₆)alkylthio, (C₁-C₆)alkylamino, heteroaryl-carbonylamino, arylcarbonylamino, (C₁-C₄)alkylsulfonylamino, di(arylsulfonyl)amino, (C₃-C₆)cycloalkylcarbonylmethyl or amino(hydroxyimino)methyl.

In certain embodiments of the invention, R² is hydrogen, R³ is hydrogen, A is sulfur, and Z is halogen, formyl, carbamoyl, cyano, trifluoromethyl, trifluoromethoxy, nitro, amino, formamido, acetamido, (C₁-C₆)alkyl, (C₁-C₆)alkoxy, (C₁-C₆)alkylthio, or (C₁-C₆)alkylamino.

In certain embodiments of the invention, Z is heteroaryl-carbonylamino, arylcarbonylamino, (C₁-C₄)alkylsulfonylamino, di(arylsulfonyl)amino, (C₃-C₆)cycloalkylcarbonylmethyl or amino(hydroxyimino). In certain other embodiments, Z is halogen, cyano, trifluoromethyl, trifluoromethoxy, methyl, ethyl, methoxy, or ethoxy.

In a particular embodiment of the invention, the compound of formula (I) is (R)-7-chloro-N-(quinuclidin-3-yl)benzo[b]thiophene-2-carboxamide, or a salt or solvate thereof, e.g., a hydrochloride salt, a monohydrate, or a combination thereof.

### Additional Compound Forms:

Certain embodiments of formula (I) may contain asymmetric centers and exist as different enantiomers or diastereomers. Some compounds of the disclosure may have one or more chiral centers and/or geometric isomeric centers (E- and Z-isomers), and it is to be understood that the present disclosure encompasses all such optical, diastereoisomers and geometric isomers. The disclosure also comprises all tautomeric forms of the compounds disclosed herein. All enantiomers or diastereomeric forms are intended to be included within the scope of the present invention. The compounds of the invention may be racemic, or in a single enantiomer form

Compounds in the disclosure may be in the form of pharmaceutically acceptable salts. The phrase “pharmaceutically acceptable” refers to salts prepared from pharmaceutically acceptable non-toxic bases and acids, including inorganic and organic bases and inorganic and organic acids. Salts derived from inorganic bases include lithium, sodium, potassium, magnesium, calcium and zinc. Salts derived from organic bases include ammonia, primary (e.g. Tromethamine), secondary and tertiary amines, and amino acids (e.g. Lysine). Salts derived from inorganic acids include sulfuric, hydrochloric, phosphoric, methanesulphonic, hydrobromic. Salts derived from organic acids include C₁₋₆ alkyl carboxylic acids, di-carboxylic acids and tricarboxylic acids such as acetic acid, propionic acid, fumaric acid, maleic acid, succinic acid, tartaric acid, adipic acid and citric acid, and alkylsulfonic acids such as methanesulphonic, and aryl sulfonic acids such as para-tolouene sulfonic acid and benzene sulfonic acid. For detailed list of slats see P. H. Stahl and C. G. Wermuth (eds.) “Handbook of Pharmaceutical Salts, Properties, Selection and Use” Wiley-VCH (ISBN 3-906390-26-8)

Compounds and pharmaceutically acceptable salts thereof may be in the form of a solvate. This occurs when a compound of the invention crystallizes in a manner that it incorporates solvent molecules into the crystal lattice. Examples of solvents forming solvates are water (hydrates), MeOH, EtOH, iPrOH, and acetone. Compounds of the invention described herein cover all solvates of the depicted compounds.

Compounds in the disclosure may exist in different crystal forms known as polymorphs.

Practitioners of the art will recognize that certain chemical groups may exist in multiple tautomeric forms. The scope of this disclosure is meant to include all such tautomeric forms. For example, a tetrazole may exist in two tautomeric forms, 1-H tetrazole and a 2-H tetrazole. This is depicted in figure below. This example is not meant to be limiting in the scope of tautomeric forms.

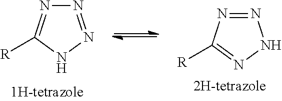

Practitioners of the art will also recognize that certain electrophilic ketones, may exist in a hydrated form. The scope of this disclosure is to include all such hydrated forms. For example, a trifluoromethyl ketone may exist in a hydrated form via addition of water to the carbonyl group. This is depicted in figure below. This example is not meant to be limiting in the scope of hydrated forms.

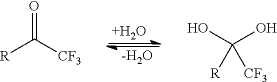

The present disclosure also includes prodrugs of compounds of the disclosure. The term “prodrug” is intended to represent covalently bonded carriers, which are capable of releasing the active ingredient when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of the disclosure include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like.

### III. METHODS OF THE INVENTION

In another embodiment, the invention provides a method for improving and/or treating a Limited Cognitive Impairment (LCI) comprising administering to a subject a therapeutically effective amount of a compound of formula (I), or a pharmaceutically acceptable salt thereof,

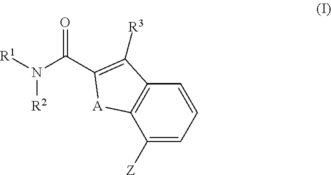

wherein

R¹ is 1-azabicyclo[2.2.2]oct-3-yl,

R² is hydrogen or (C₁-C₆)alkyl,

R³ is hydrogen, halogen or (C₁-C₆)alkyl,

A is oxygen or sulfur, and

Z is halogen, formyl, carbamoyl, cyano, trifluoromethyl, trifluoromethoxy, nitro, amino, formamido, acetamido, (C₁-C₆)alkyl, (C₁-C₆)alkoxy, (C₁-C₆)alkylthio, (C₁-C₆)alkylamino, heteroaryl-carbonylamino, arylcarbonylamino, (C₁-C₄)alkylsulfonylamino, di(arylsulfonyl)amino, (C₃-C₆)cycloalkylcarbonylmethyl or amino(hydroxyimino)methyl.

In another embodiment, the subject is treated with a compound of formula (I) wherein R² is hydrogen, R³ is hydrogen, A is sulfur, and Z is halogen, formyl, carbamoyl, cyano, trifluoromethyl, trifluoromethoxy, nitro, amino, formamido, acetamido, (C₁-C₆)alkyl, (C₁-C₆)alkoxy, (C₁-C₆)alkylthio, or (C₁-C₆)alkylamino.

In another embodiment, the subject is treated with a compound of formula (I) wherein Z is heteroaryl-carbonylamino, arylcarbonylamino, (C₁-C₄)alkylsulfonylamino, di(arylsulfonyl)amino, (C₃-C₆)cycloalkylcarbonylmethyl or amino(hydroxyimino).

In another embodiment, the subject is treated with a compound of formula (I) wherein Z is halogen, cyano, trifluoromethyl, trifluoromethoxy, methyl, ethyl, methoxy, or ethoxy.

In another embodiment, the subject is treated with a compound of formula (I) wherein the compound of formula (I) is (R)-7-chloro-N-(quinuclidin-3-yl)benzo[b]thiophene-2-carboxamide.

In certain embodiments, the subject has been diagnosed with a symptom, condition, or disorder classified as a Limited Cognitive Impairment. In particular embodiments, the methods of administering a compound of the invention described herein improve one or more cognitive impairments (i.e., symptoms or conditions), which precede AD on a clinical diagnostic scale but are not of such an increased degree to be quantitatively or qualitatively classified as mild cognitive impairment; and includes any chronic or temporary impairment in cognition, learning or memory that prevents or reduces the ability of a particular individual from achieving their individual potential capacity in these areas. For example, LCIs may include minor impairments to memory associated with focus and concentration (e.g., accuracy and speed of learning and recalling information), working memory (e.g., used in decision making and problem solving), cognition, focus, mental quickness, and mental clarity. In particular independent embodiments, subjects afflicted with an LCI are those subjects that demonstrate these minor and/or brief impairments, such as alcohol or drug (e.g., amphetamine, cannabis, cocaine, hallucinogens, inhalants, opioids, or phencyclidine) affected subjects (e.g., subjects with a hangover or the drug-induced state); subjects affected by various disorienting therapies (e.g., cancer therapy); subjects that are sleep deprived (e.g., shift workers, or frequent or international travelers); subjects that exhibit one or more symptoms of attention deficit disorder (ADD) or attention deficit hyperactivity disorder (ADHD), but are undiagnosed as ADD or ADHD due to failure to fully qualify under strict definitions of these disorders; subjects with minor post operative or anesthetic induced disorientation or mental fog; or subjects affected by minor or brief postpartum memory loss or lack of concentration.

In this respect, it has been found that (R)-7-chloro-N-(quinuclidin-3-yl)benzo[b]thiophene-2-carboxamide can have beneficial effects in humans at unexpectedly low doses. As such, (R)-7-chloro-N-(quinuclidin-3-yl)benzo[b]thiophene-2-carboxamide and pharmaceutically acceptable salts thereof can be used at unexpectedly low doses improve cognition in individuals suffering from Limited Cognitive Impairment at a daily dose of 3 mg, 2.70 mg, 2.50 mg, 2.25 mg, 2 mg, 1.75 mg, 1.50 mg, 1.25 mg, 1 mg, 0.7, 0.5, 0.3 mg or even 0.1 mg. The compound can be used to improve one or more aspects of cognition related to LCI, for example, minor impairments to memory associated with focus and concentration (e.g., accuracy and speed of learning and recalling information), working memory (e.g., used in decision making and problem solving), cognition, focus, mental quickness, and mental clarity. In a particular embodiment, (R)-7-chloro-N-(quinuclidin-3-yl)benzo[b]thiophene-2-carboxamide or a pharmaceutically acceptable salt thereof is administered at 0.03 to 1.0 mg/day. In a particular embodiment, (R)-7-chloro-N-(quinuclidin-3-yl)benzo[b]thiophene-2-carboxamide or a pharmaceutically acceptable salt thereof is orally administered at 0.03 to 0.5 mg/day. In a particular embodiment, (R)-7-chloro-N-(quinuclidin-3-yl)benzo[b]thiophene-2-carboxamide or a pharmaceutically acceptable salt thereof is orally administered at 0.03 to 0.3 mg/day. In a particular embodiment, (R)-7-chloro-N-(quinuclidin-3-yl)benzo[b]thiophene-2-carboxamide or a pharmaceutically acceptable salt thereof is orally administered at 0.03 to 0.1 mg/day.

The compounds of the invention, e.g., compound of formula (I), can be administered in any convenient manner, e.g., orally, or transdermally.

Combinations

In certain embodiments of the invention, the compound of formula (I) may be administered in combination with an acetylcholinesterase inhibitor. In certain embodiments, the compound of formula (I) is administered at a dose that is therapeutically effective in the absence of an acetylcholinesterase inhibitor. It has also been surprisingly found that certain aspects of cognition can be improved when (R)-7-chloro-N-(quinuclidin-3-yl)benzo[b]thiophene-2-carboxamide is administered at a subclinical dose (i.e., a dose that but for the combination would not improve memory or cognition) in combination with an acetylcholinesterase inhibitor that is also administered at a subclinical dose. Thus, a subject can experience a benefit (e.g., improved memory or cognition) from a combination of drugs each of which is administered at very low, side-effect reducing or side-effect avoiding dose. Moreover, the combination of drugs may provide a benefit for a wider range or subjects and/or over a longer period of treatment. For example, while certain acetylcholinesterase inhibitors can exhibit reduce efficacy after several months of treatment, the combination may provide a longer period of efficacy.

Accordingly, the present invention provides for the further combination of an alpha7 agonist described herein (e.g., (R)-7-chloro-N-(quinuclidin-3-yl)benzo[b]thiophene-2-carboxamide), and an acetylcholinesterase inhibitor, wherein either the compound of formula (I), the acetylcholinesterase inhibitor, or both are administered at a subclinical dose. In certain embodiments, the subject has been diagnosed with and LCI. In particular embodiments, the acetylcholinesterase inhibitor is selected from tacrine, donepezil, rivastigmine and galantamine, e.g., donepezil. In certain embodiments wherein the acetylcholinesterase inhibitor is selected from donepezil, rivastigmine and galantamine, the subject has been administered an acetylcholinesterase inhibitor for a period of time prior to being administered (R)-7-chloro-N-(quinuclidin-3-yl)benzo[b]thiophene-2-carboxamide or a pharmaceutically acceptable salt thereof, wherein the prior administration has been for at least one month, e.g., the prior administration has been for at least three months, e.g., the prior administration has been for at least six months.

In particular embodiments, (R)-7-chloro-N-(quinuclidin-3-yl)benzo[b]thiophene-2-carboxamide or a pharmaceutically acceptable salt thereof is orally administered at 1.0 mg/day; 0.5 mg/day; 0.3 mg/day; or 0.1 mg/day. In particular embodiments, the acetylcholinesterase inhibitor is donepezil and is orally administered at 5 mg/day; 4.5 mg/day; 4.0 mg/day; 2.5 mg/day; 1.5 mg/day or less; 1.0 mg/day; and the acetylcholinesterase inhibitor is administered at a dose that achieves 10-65% steady state red blood cell acetylcholinesterase inhibition.

As disclosed herein, individual components of the combinations useful in this invention may generally be administered separately, each by its own customary and known route; and in certain cases the routes of administration may be different. In a particular embodiment, administration will generally be timed so that both the compound of formula (I), and the acetylcholinesterase inhibitor both coincide, or nearly coincide, in reaching their maximum pharmacokinetic effect. The routes of administration can be any of those known to the art such as oral, parenteral via injection, or transdermal as by applying the active component in a gel, a patch, or other such formulation topically. Each component can be formulated as known in the art, usually together with a pharmaceutically acceptable vehicle, diluent or carrier, for example as a tablet, capsule, lozenge, troche, elixir, solution, or suspension for oral administration, in a suitable injectable vehicle for parenteral administration, or as a lotion, ointment or cream for topical application. In a particular embodiment, the compound of formula (I) and acetylcholinesterase inhibitor are each co-administered orally, together or separately.

The exact dose of each component administered will, of course, differ depending on the specific components prescribed, on the subject being treated, on the severity of the disorder, on the manner of administration and on the judgment of the prescribing physician. Thus, because of subject-to-subject variability, the dosages given below are a guideline and the physician may adjust doses of the compounds to achieve the treatment that the physician considers appropriate for the subject, male or female. In considering the degree of treatment desired, the physician must balance a variety of factors such as the age of the subject and the presence of other diseases or conditions. In general, a compound of formula (I) can be used at low doses, for example at a daily oral dose of (or no more than): 3 mg, 2.70 mg, 2.50 mg, 2.25 mg, 2 mg, 1.75 mg, 1.50 mg, 1.25 mg, 1 mg, 0.7 mg, 0.5 mg, 0.3 mg, 0.1 mg, 0.5 mg or even 0.03 mg. Thus, for example, it can be administered at 0.03-1.5 mg, 0.05-1.5 mg, 0.05-1.0 mg daily dose, including 1 mg/daily, 0.5 mg/daily or 0.3 mg/daily. In some cases the dose of (R)-7-chloro-N-(quinuclidin-3-yl)benzo[b]thiophene-2-carboxamide that is administered is a subclinical dose.

### IV. PHARMACEUTICAL COMPOSITIONS OF THE INVENTION

In one embodiment, the present invention provides a pharmaceutical composition comprising a therapeutically effective amount of (R)-7-chloro-N-(quinuclidin-3-yl)benzo[b]thiophene-2-carboxamide, or a pharmaceutically acceptable salt thereof, useful for improving or treating LCI in a subject.

Another embodiment of the present invention provides a packaged pharmaceutical comprising a package containing a unit dosage pharmaceutical composition comprising (R)-7-chloro-N-(quinuclidin-3-yl)benzo[b]thiophene-2-carboxamide or a pharmaceutically acceptable salt thereof, and instructions for use in improving or treating LCI. In one embodiment, the packaged pharmaceuticals may comprise a therapeutically effective amount of (R)-7-chloro-N-(quinuclidin-3-yl)benzo[b]thiophene-2-carboxamide or a pharmaceutically acceptable salt thereof, useful for improving or treating LCI in a subject. In some cases the unit dosage form of (R)-7-chloro-N-(quinuclidin-3-yl)benzo[b]thiophene-2-carboxamide or a pharmaceutically acceptable salt thereof is a tablet or capsule. In particular embodiments, the package further comprises instructions for use, e.g., describing a regimen of treatment using the compound of formula (I) for the treatment of LCI, and such instructions may optionally form an integrated component of the packaging.

In certain embodiments, the pharmaceutical composition may also comprise an acetylcholinesterase inhibitor. As such, a particular embodiment, the present invention provides a pharmaceutical composition comprising (R)-7-chloro-N-(quinuclidin-3-yl)benzo[b]thiophene-2-carboxamide or a pharmaceutically acceptable salt thereof, and an acetylcholinesterase inhibitor. In certain embodiments, the acetylcholinesterase inhibitor is selected from tacrine, donepezil, rivastigmine and galantamine, e.g., donepezil.

Also described is a daily unit dosage pharmaceutical composition comprising no more than 1.0 mg of (R)-7-chloro-N-(quinuclidin-3-yl)benzo[b]thiophene-2-carboxamide or a pharmaceutically acceptable salt thereof, an acetylcholinesterase inhibitor and a pharmaceutically acceptable carrier. In various cases the daily unit dosage pharmaceutical composition comprises no more than 0.5 (0.3, or 0.1) mg of (R)-7-chloro-N-(quinuclidin-3-yl)benzo[b]thiophene-2-carboxamide or a pharmaceutically acceptable salt thereof. In certain embodiments, the daily unit dosage pharmaceutical composition comprises no more than 5, 4, 3, 2, 1, or 0.5 mg of donepezil.

For oral administration, a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; in particular embodiments, such materials also include lactose or milk sugar as well as high molecular weight polyethylene glycols. When aqueous suspensions and/or elixirs are desired for oral administration, the compounds/components of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, may be prepared.

Methods of preparing various pharmaceutical compositions with a certain amount of active ingredient are known, or will be apparent in light of this disclosure, to those of ordinary skill in this art. For examples of methods of preparing pharmaceutical compositions, see Remington's Pharmaceutical Sciences, Mack Publishing Company, Easter, Pa., 15th Edition (1975). After a pharmaceutical composition has been formulated in an acceptable carrier, it can be placed in an appropriate container and labeled for treatment of an indicated condition. For administration of the compounds of the invention, such labeling would include, e.g., instructions concerning the amount, frequency and method of administration.

### EXEMPLIFICATION

The present invention is illustrated by the following examples, which are not intended to be limiting in any way.

Compounds of the invention may be synthesized according to art-recognized techniques and synthetic strategies.

### Example 1

### General Safety and Efficacy Assessment

The safety and efficacy for the compounds of the invention may be assessed in accordance with existing strategies for such assessment. For example, the safety and efficacy of (R)-7-chloro-N-(quinuclidin-3-yl)benzo[b]thiophene-2-carboxamide HCl salt can be assessed in subjects, e.g., subjects suffering from LCI as follows. Subjects are dosed with placebo or (R)-7-chloro-N-(quinuclidin-3-yl)benzo[b]thiophene-2-carboxamide HCl salt at appropriate doses. Safety can be evaluated by adverse events, ECG, and clinical laboratory measures. Cognitive effects can be measured by CogState computerized cognitive testing and all or a subset of NTB scales (e.g., category fluency, Trails A and B).

### Example 2

### Effect on Cognition in LCI Subjects

The studies described below are able to demonstrate that (R)-7-chloro-N-(quinuclidin-3-yl)benzo[b]thiophene-2-carboxamide hydrochloride can improve sensory electrophysiological responses which correlate with improved cognitive and functional performance in LCI subjects in manner that is greater than placebo. These effects are expected at a daily dose as low as 0.3 mg of (R)-7-chloro-N-(quinuclidin-3-yl)benzo[b]thiophene-2-carboxamide hydrochloride.

Impairment of the ability of central nervous system to inhibit irrelevant sensory information has long been used as a model for understanding the deficits of attention seen in subjects. Two approaches to the measurement of this ability have commonly been employed (see (Heinrichs, 2004; Potter et al., 2006; Turetsky et al., 2007; Umbricht and Krljes, 2005) for reviews and meta-analyses): (1) the sensory gating paradigm in which the presentation of one stimulus normally suppresses the response elicited by a stimulus which rapidly follows it; and (2) the oddball or orienting paradigm in which a rare or unexpected event elicits a diminished response in subjects because attentional resources are inappropriately focused on less salient aspects of the environment.

Two responses are commonly used assess brain activity: (1) the auditory P50 response elicited by the second member of a pair of clicks; and (2) the mismatch negativity (MMN) or N2 response evoked by a rarely occurring pure tone of no instructed relevance to the subject. Abnormalities in both P50 gating and the MMN have been reported in schizophrenic subjects.

The neurobiology of P50 sensory gating is well documented in studies of human and animal subjects. Its regulation relies heavily on the integrity of the hippocampus and pathways that provide input to the hippocampus (Adler et al., 1998). For example, lesions of the cholinergic pathway originating in the medial septal nucleus disrupt the gating response, as do antagonists of low affinity nicotinic receptors. Cholinergic agonists, including nicotine itself (Adler et al., 1993; Duncan et al., 2001), have been shown to enhance P50 gating (Freedman et al, 2001; Olincy et al., 2006).

The neurobiology of the MMN is more complex. Imaging studies suggest that the primary and secondary auditory cortices in the temporal lobe are important for its generation (Naatanen and Alho, 1995). The dorsolateral prefrontal cortex also contributes (Schall et al., 2003). The neurotransmitter systems underlying the MMN are understudied and largely unknown. Yet, as is the case for P50, nicotinic cholinergic systems appear important (Baldeweg et al., 2006; Dunbar et al., 2007).

The sensitivity of P300 and N100 to cholinergic compounds has been known for many years (Dierks et al., 1994; Kaga et al., 1992). Various cholinergic antagonists—such as scopolamine—profoundly reduce the amplitudes of these components. In contrast, the components are markedly improved in amplitude by cholinesterase inhibitors (Katada et al., 2003; Werber et al., 2001) and other compounds that enhance cholinergic activity (Easton and Bauer, 1997).

The tests described above are used to study the effect of the test compound or combination, in comparison to placebo, on cognition in subjects suffering from LCI. Prior to testing in the assays described above, the subjects are dosed with 1 mg of a compound of formula (I) daily, 0.3 mg of a compound of formula (I) daily or placebo for 20 days.

### Example 3

### Effect on Cognition in Normal Subjects

The impact of the compound of formula (I), e.g., (R)-7-chloro-N-(quinuclidin-3-yl)benzo[b]thiophene-2-carboxamide hydrochloride, on cognition in normal subjects may be assessed as described below. In these studies subjects are treated with the compound dissolved in cranberry juice.

The impact of the compound on cognition in normal subjects is assessed in an SAD (Single Ascending Dose) study with the Digit Symbol Substitution Test (DSST). Positive effects of a test compound in the DSST indicate a beneficial effect on working memory and executive function.

In the MAD (Multiple Ascending Dose) studies cognition is assessed using tests from the CogState battery (cogstate.com). The CogState battery is a proprietary computerized cognitive battery of tests measure various cognitive domains including: attention, identification capability, working memory, visual memory, and executive function. In these studies a test compound is evaluated for an impact on: visual motor skills, learning, executive function, and delayed memory. A therapeutic profile showing pro-cognitive effects on nonverbal learning and memory and executive function without a central stimulatory effect would indicate that the drug may be very beneficial in treating subjects that have, as a feature of their condition, symptoms of anxiety or agitation.

### INCORPORATION BY REFERENCE

The entire contents of all patents, published patent applications and other references cited herein are hereby expressly incorporated herein in their entireties by reference.

### EQUIVALENTS

Those skilled in the art will recognize, or be able to ascertain using no more than routine experimentation, numerous equivalents to the specific procedures described herein. Such equivalents were considered to be within the scope of this invention and are covered by the following claims. Moreover, any numerical or alphabetical ranges provided herein are intended to include both the upper and lower value of those ranges. In addition, any listing or grouping is intended, at least in one embodiment, to represent a shorthand or convenient manner of listing independent embodiments; as such, each member of the list should be considered a separate embodiment.

1-19. (canceled)

20. A method of improving and/or treating Limited Cognitive Impairment (LCI) in a patient in need thereof, comprising administering to the patient a therapeutically effective amount of a compound of formula (I), or a pharmaceutically acceptable salt thereof, 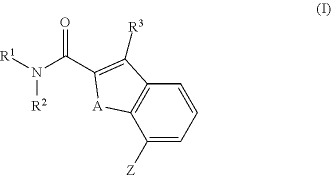
wherein
R¹ represents I-azabicyclo[2.2.2]oct-3-yl,
R² represents a hydrogen or (C₁-C₆)alkyl,
R³ represents a hydrogen, halogen or (C₁-C₆)alkyl,
A represents an oxygen or sulfur, and
Z represents a halogen, formyl, carbamoyl, cyano, trifluoromethyl, trifluoromethoxy, nitro, amino, formamido, acetamido, (C₁-C₆)alkyl, (C₁-C₆)alkoxy, (C₁-C₆)alkylthio, (C₁-C₆)alkylamino, heteroaryl-carbonylamino, arylcarbonylamino, (C₁-C₄)alkylsulfonylamino, di(arylsulfonyl)amino, (C₃-C₆)cycloalkylcarbonylmethyl or amino(hydroxyimino)methyl.

21. The method of claim 20, wherein the patient has been diagnosed with a symptom, condition, or disorder classified as Limited Cognitive Impairment.

22. The method of claim 20, wherein the method improves one or more of the following minor impairments: impairment to memory associated with focus and concentration, impairment to working memory, impairment to cognition, impairment to focus, impairment to mental quickness, and impairment to mental clarity.

23. The method of claim 20, wherein the method improves cognition in the patient.

24. The method of claim 20, wherein the compound of formula (I) is (R)-7-chloro-N-(quinuclidin-3-yl)benzo[b]thiophene-2-carboxamide.

25. The method of claim 24, wherein the administered therapeutically effective amount is administered at 0.1 to 3.0 mg/day.

26. The method of claim 24, wherein the administered therapeutically effective amount is administered at 0.03 to 1.0 mg/day.

27. The method of claim 24, wherein the administered therapeutically effective amount is administered at 0.03 to 0.5 mg/day.

28. The method of claim 24, wherein the administered therapeutically effective amount is administered at 0.03 to 0.3 mg/day.

29. The method of claim 24, wherein the administered therapeutically effective amount is administered at 0.03 to 0.1 mg/day.
